(19) United States
(12) Patent Application Publication
(10) Pub. No.: US 2022/0223246 A1
(43) Pub. Date: Jul. 14, 2022

(54) METHOD AND SYSTEM FOR PERSONALIZING AND STANDARDIZING CANNABIS, PSYCHEDELIC AND BIOACTIVE PRODUCTS

(71) Applicant: APOTHECA SYSTEMS INC., Vancouver (CA)

(72) Inventor: Christopher Craig BOOTHROYD, Vancouver (CA)

(21) Appl. No.: 17/614,485

(22) PCT Filed: May 29, 2020
(86) PCT No.: PCT/CA2020/050745

§ 371 Date: Nov. 26, 2021

Related U.S. Application Data
(60) Provisional application No. 62/855,271, filed on May 31, 2019.

(51) Int. Cl. G16H 20/10; G06Q 30/06; G06Q 30/02

(52) U.S. Cl. CPC G16H 20/10; G06Q 30/0203; G06Q 30/0631

(57) ABSTRACT

A computer-implemented method to establish and maintain standards for bioactive products such as cannabis according to predicted consumer outcome is provided. Consumers and producers interactively communicate with a knowledge graph database organized according to specified product formulations, methods of delivery of the bioactive products, and surveyed consumer outcomes with each bioactive product formulation. By curating the knowledge graph database of bioactive product formulations standardized by product formulation, product delivery method and consumer outcome organized by cohorts according to consumer profiles, a consumer can be provided a recommended formulation of the bioactive product which will provide the desired outcome.

### REFERENCE TO RELATED APPLICATIONS

The present application claims the benefits, under 35 U.S.C. § 119(e), of U.S. Provisional Application Ser. No. 62/855,271 filed May 31, 2019 entitled “METHOD AND SYSTEM FOR DIGITALLY STANDARDIZING CANNABIS PRODUCTS ACCORDING TO PREDICTED CONSUMER EXPERIENCE” which is incorporated herein by this reference.

### TECHNICAL FIELD

The invention relates to the application of data-analytical and theoretical methods, mathematical modeling and computational simulation techniques to the study, analysis and testing of cannabis, psychedelic and other bioactive products for purposes of defining, classification and standardization of evidence-based formulations for consumer personalization.

### BACKGROUND

The Cannabis sativa plant, or marijuana, produces a number of unique organic compounds including cannabinoids and terpenes or terpinoids. The primary psychoactive compound is tetrahydrocannabinol (THC). THC and at least 65 other chemical compounds are unique to the cannabis plant. These include cannabichromene (CBC), cannabicyclol (CBL), cannabidiol (CBD), cannabielsoin (CBE), cannabigerol (CBG), cannabinidiol (CBND), cannabinol (CBN), cannabitriol (CBT) and cannabichromanone (CBCN). Cannabis products are used in many ways, including inhalation (smoking, vaporizing or vaping), eating as part of edible food products, and in extracts such as hashish, kief, tinctures, infusions in solvents and oils.

Currently marijuana and cannabis-based products are legal for medical purposes in many jurisdictions and also legal for recreational use in some of those jurisdictions. For many years however cannabis was an illegal black market product, so minimal published research on cannabis products has been done until recently.

Due to the legal status of cannabis, little shared or peer-reviewed experimentation has been possible. There is no standardization of different strains among producers currently, for example. Consumers however increasingly want to know what effects a particular cannabis product will provide. For example, cannabis consumers want to know how a specific strain will make them feel, or whether it will have specific positive or negative medical or physiological effects. Even medical cannabis data lacks standardization for the patients. Currently certain brands will associate their product with specific effects, however cannabis affects different people in different ways. Since brand manufacturers are profit-motivated, a testing bias towards a marketing target demographic has an effect on the results, which may be inaccurate.

The first challenge with classifying or standardizing cannabis products stems from the wide variety of cannabinoid and terpene compounds and their relative proportions which may occur in any given batch of a product. The interactive synergy among different cannabis compounds creates a complex reaction in humans coined as the “entourage effect”. Producers currently display a ratio of THC and CBD on product labels, but this is an often inaccurate estimate and does not specify presence of other cannabinoids or terpenes. For cannabis products, there has been no vendor-independent effort to label an exact formulation, as would be required for ISO certification. Producers are uncertain of the exact formulation obtained from any given source. Unscientific methods are often currently in use for estimations of THC and other component concentrations. Even when the source is lab measured, production conditions make it difficult or expensive to maintain exact component concentrations, and ranges of error must be provided, with often up to a 50% error margin.

The second challenge is that the effect of a given cannabis product on a given user varies widely depending on a large number of factors (“Co-factors”). These include the method of delivery, the user's current physical and mental state (lack of sleep, hungry, happy, depressed, sick, obese, recent consumption of alcohol, caffeine, nicotine or other drugs), the user's sex and age, the user's genetic make-up, ancestry, DNA, and the like. The effect of such factors for a given individual are not known in the absence of extensive clinical trials and even then, it is likely that some co-factors are not considered or recorded, so even with the same formulations, exact subjective outcome repeatability is elusive. Modern pharmaceutical studies do not have a set of experimental templates for cognitive subjective outcomes for recreational drugs. Alcohol and nicotine are prime examples. Added to the problem with cannabis is that due to prohibition, little experimentation has been possible. Other factors that have not been taken into account are hereditary genetic drift between CB1 and CB2 receptor activity, and other biochemical and genetic factors that are still to be discovered after applying analysis of big data. Other co-factors that have not been studied in connection with subjective cannabis outcomes include non-cannabinoids and non-terpenes including coffee, alcohol and sugars, DNA, sex, age, diet, and the like. No reliable framework for quantifying subjective outcomes to provide an evidence-based formulation has therefore been developed.

Similar problems in standardization exist with psychedelics, hallucinogens and what are sometimes referred to as entheogens. Similar to marijuana, psychedelic drugs such as LSD, ibogaine, psilocybin, mescaline, ketamine, DMT, MDMA, 2C-B, 2C-I, 5-MeO-DMT, AMT, and DOM have long shown promise for treatment of various medical conditions including opioid and other addictions, alcoholism, depression, anxiety, obsessive compulsive disorder and post-traumatic stress syndrome. As used herein the term “psychedelic drug” includes 5-HT2A agonists (e.g., lysergic acid diethylamide or psilocybin), dissociative agents (e.g., ketamine), and empathogenic agents such as MDMA. Considerable medical research was done in the 1950's and 1960's for some of these drugs, such as LSD, however like marijuana, medical research respecting such drugs essentially ceased since the 1970's due to their illegality. Recently there has been renewed interest in clinical research in treatment using psychedelics. One current form of therapy using psychedelics involves low dosing or micro-dosing. However, similar to cannabis-based products, the lack of significant clinical research and the fact that the effects of psychedelic drugs on the human mind are very complex, highly individualized and difficult to categorize makes it almost impossible for a physician to confidently prescribe the correct psychedelic drug and dosage for a particular patient, should medical uses become legal.

Traditionally to establish the validity and efficacy of a drug, clinical trials are conducted to discover and validate typically a single molecule that is tested for efficacy outcomes against a target condition (such as arthritic pain for example). These tests are statistically driven studies of a carefully built sample set of individuals that represent the population, that upon drug validation, can give a statistically meaningful probability of population majority efficacy success against that target condition. This however is imperfect as no two individuals share the exact same gene/body/mind profile and statistically there is no accounting for this factor. The problem is even after drug approval and a high coefficient of study success, there is still no adequate measure against a known patient's complete and unique profile, and drug manufacturers and regulatory bodies are relying on the statistical correlation strength of the clinical trial to deliver efficacy to the patient. In this fashion, most prescriptions and treatment plans are created by a health professional, with the available knowledge they have from the currently available patient health records, lab tests and literature to make the best educated guess they can about the outcome of prescribing that drug for that patient who has never taken that drug before. In recent years Precision Medicine has begun to address the need to apply the individual's complete gene/body/mind profile to the equation.

Bioinformatics is a branch of information science covering research, development, or application of computational tools and approaches for expanding the use of biological, medical, behavioral or health data, including computational tools to acquire, store, organize, archive, analyze, or visualize such data. A related field is Computational Biology, which is the science of using biological data to develop algorithms or models to understand biological systems and relationships. Biologists now have access to very large amounts of data and using Bioinformatics and Computational Biology can interpret such data, which is particularly useful in molecular biology and genomics. Machine learning techniques are also available to manage the organization and analysis of very large amounts of data.

There is therefore a general need for a method to apply bioinformatics and machine learning methods to permit individual consumers to map themselves to obtain a selected cannabis product or similar product, independent of the producer's promotion and claims, to obtain a specific desired outcome from the cannabis product according to the user's profile and current state of mind and body, and chosen method of delivery (“the Co-factors”). There is a need for consumers to map themselves to a range of cannabis products to get expected results, and to be mapped to cohorts for cohort outcome benefits and statistical analysis. This requires a fixed set of formulation points that do not change and can be reliably targeted by producers on one side and by consumers on the other. What is required is a method to unify all producers' clinics and using the equivalent of clinical trials to produce large amounts of organized data covering safety, efficacy and likely effects of cannabis products on a given individual before they are approved for consumer sale using an ISO-like certification standard. The foregoing general need also arises among consumers of psychedelic drugs and other bioactive molecular formulations, both for physicians prescribing such drugs for medical treatment and users of such drugs for both medical and recreational use.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

While the invention described as follows has been found to be particularly suited for cannabis products, it is similarly applicable to psychedelics, entheogens and other bioactive molecular formulations and the methods described below should be understood to apply in the same way to psychedelics, entheogens and other bioactive molecular formulations as they do to cannabis products. The results are achieved through application of principles of Personalized or Precision Medicine, and Evidence-based Formulations through the application of Artificial Intelligence to provide consumers the outcomes they need from cannabis, psychedelics and other bioactive molecular formulations.

The invention provides a method to use Formulation/Cohort Points to allow a consumer to interpolate and predict which cannabis formulations will provide a predictable, certifiable effect for that individual consumer. This is achieved by collecting personal data and subjective outcome data from increasingly large samples of cannabis consumers and using bioinformatics and machine learning to analyze patterns from a large number of inputs to create a self-correcting expanding Knowledge Graph database whereby users can share personalizations in like-outcome cohorts to enable leveraging of an expanded range of analysis methods. While a user can only relate their own outcome, statistically grouping similar outcomes generates a reproducible prediction of the effects of the cannabis product.

One aspect of the invention provides a Knowledge Graph/Formulation database based on a set of knowns including a complete set of possible formulations across Cannabinoids/Terpenes, referred to herein as Formulations (“FNs”), against which the individual consumer can map his or her Co-Factors. Formulations may have properties defined across many dimensions such as component concentration, and co-factor variables. Formulations provide a consumable defined outcome mapping space between cannabis product sources and people. Formulations not only create fixed repeatable points in cannabis subjective outcome space but can also be used to create bounded outcome volumes that source, people and co-factors can be mapped into. Bounded volumes may also create data value in their intersectional overlap with other bounded volumes.

The invention therefore provides a computer-implemented method to establish and maintain standards for cannabis products according to predicted consumer outcomes, wherein cannabis consumers and producers are provided with computer devices for accessing and interactively communicating via a computer network with a system server for managing a Knowledge Graph database of cannabis strains organized according to possible cannabis product formulations, methods of cannabis delivery, certified product identification and surveyed consumer outcomes with each cannabis product formulation and delivery method against which one of the consumers can map his or her profile and desired outcome to obtain a recommended cannabis product for a desired outcome, the method comprising: a) receiving from a plurality of cannabis producers a plurality of defined cannabis products of the producers; b) defining a set of unique cannabis product formulations; c) curating a knowledge graph database of cannabis product formulations standardized by product formulation, cannabis delivery method and consumer outcome organized by cohorts according to consumer profiles; d) receiving from a plurality of consumers a plurality of consumer profiles and proposed product outcomes for each consumer; e) processing a recommended product for each consumer; f) receiving some or all of said consumers who have received recommendations a description of the outcome received from said recommended cannabis product; g) defining a range of population cohorts based on common profile characteristics; h) associating each consumer who has provided an outcome description to one or more of the population cohorts; i) curating the knowledge graph database to link outcomes of said consumers to the associated cohort; and j) using machine learning applications to curate the knowledge graph to improve the accuracy of said outcome predictions.

According to one aspect the cannabis product formulations may comprise relative percentages of cannabinoids. The Formulation profile may comprise factors selected from the group consisting of formulation, delivery, outcome cohorts and products. The consumer profile may comprise factors selected from the group consisting of genetic, phenotype, health, cognitive, environment and social. The producers' defined cannabis products may be provided a cannabis product certification. The certified cannabis products may be associated with an accurate prediction for the effect of such product on a population cohort and/or be provided a standardized grade for commodity transactions, such as cannabis asset-secured transactions. Iconography or QR code may be associated with cannabis products so a user can be quickly directed to the certification information. The invention may provide a system for carrying out the described method such as a data marketplace wherein consumers who have provided outcome descriptions and the curators of the Knowledge Graph database are compensated for the sale or use of said data.

According to a further aspect the method of the invention includes the following steps.
A. Formulation: The first step is to force refinement and analysis of natural and entheogenic compounds using laboratory techniques to create a set of fixed formulation component values, so that one knows exactly what is being consumed and have a statistically relevant variable to use in prediction, so there is a known formulation standard for a particular compound. Each of these is defined as a discrete Formulation entity for the collection of tester data and efficacy extrapolation.
B. Individual Biological Profile: The second step is to provide a system where the individual's biological profile can be created, tracked and updated across an acceptable and ongoing biological state interval, so that the health practitioner and statistical analytical methods always have the latest and best data available to choose and predict a drug for treatment of a condition.
C. Knowledge—The third step is a system of condition topic knowledge aggregation that includes research, individual biological profiles, formulations, predictions and active studies that are curated by human knowledge workers and AI mediated content selection. Both contribute to the curation of the knowledge graph. This information may be queried and presented to the Health Practitioner and individual through a Bot-mediated User Interface that will create a set of relevant condition knowledge objects to help select the set of condition treatment drug options.
D. Population vs. Individual Drug Design—While modern pharmaceuticals use very well defined formulation standards for drugs that have successfully passed the clinical trial efficacy method for a given condition, there still exists a statistically significant gap in the ability to accurately predict the exact outcome efficacy for an individual with the same condition. The fourth step uses the concept of a drug outcome efficacy evaluation assay set to determine the individual's biological condition response to a set of possible viable drug treatments and statistically determine which drug formulation is delivering the best condition outcome efficacy for that patient.
E. Prediction—In order to compare the individual's biological profile factors, with those of other individuals who have taken formulated drugs and achieved high efficacy outcome success or failure for that condition, as a fifth step statistical comparative analysis and machine learning is used to identify specific biological profile factors across a set of individuals with a successful/unsuccessful condition outcome efficacy and these are used to create a predictive condition outcome efficacy scoring system for each viable formulated drug option, for the health practitioner and individual to select from for treatment. This may include AI features to evaluate primary research to help score a profile factor's statistical significance and/or a specific formulation's condition efficacy to be considered.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a schematic diagram illustrating a system for digitally standardizing cannabis products.

FIG. 2 is a schematic diagram illustrating the interaction of different components within the system for digitally standardizing cannabis products.

FIG. 3 is a schematic diagram illustrating an example of a Formulation in which a product formulation is mapped onto five different cohorts.

FIG. 4 is a schematic diagram illustrating the current difficulty in linking a given cannabis consumer to a suitable and desired cannabis product.

FIG. 5 is a schematic diagram illustrating the use of a Bot to assist in the creation and updating of the Formulations Knowledge Graph Database.

FIG. 6 is a flow chart illustrating the steps in an n=1 adaptive precision research study.

FIGS. 7 to 10 are schematic diagrams illustrating case studies using four individuals.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

### Definitions

The following terms have the following meanings herein.

“Bioactive Products” means products including a compound that has an effect on a living organism, including cannabis, psychedelics, and entheogens.

“Cannabis” means the Cannabis sativa plant, or marijuana.

“THC” means the compound tetrahydrocannabinol.

“Cannabinoids” means chemical compounds, natural or synthetic, originally derived from to the cannabis plant.

“Cannabis products” mean any product containing cannabis or compounds derived from cannabis.

“Cohort” has the meaning in the context of clinical trials of a group of people who share a defining characteristic, relevant to the particular study or trial.

“Outcome Cohort” means a group of people who share a common outcome when using a specified cannabis or psychedelic drug formulation.

“Population Cohorts” mean cohorts of populations with common profile factors.

“Bot” means a computer application for gathering and organizing data, which may apply machine learning methods by conducting an interactive communication via auditory or textual methods, acting independently to perform tasks for its principal, whether a person or another computer program. In this application bots assist the user's capability to provide and organize useful information about the user or the user's products.

“Knowledge Graph” as used herein means an ordered representation of information or data such as an RDF (Resource Description Framework) graph or a Label Property Graph. An RDF graph consists of triples according to an Entity Attribute Value (EAV) model, in which the subject is the entity, the predicate is the attribute, and the object is the value. Each triple has a unique identifier known as the Uniform Resource Identifier, or URI. The parts of a triple, the subject, predicate, and object, represent the nodes and edges in a graph. It can be visually represented as a graph consisting of nodes and edges. It can consist of a number of subgraphs. The Label Property Graph is one of a few data representation approaches that is utilized in graph databases. The data is organized as nodes, relationships, and properties. A node is an entity that can have zero or more properties. Properties are key-value pairs. Finally, relationships link two nodes in a directed way. Moreover, relations may also have zero or more properties. A suitable Knowledge Graph in this application is Apache TinkerPop-enabled graph using Gremlin language.

A “Schema” is an organized set of classes, properties, and relationships organized into hierarchies or profiles.

“Psychedelic” means one of a hallucinogenic class of psychoactive drugs. natural or synthetic, whose primary action is to trigger psychedelic outcomes via serotonin receptor agonism causing specific psychological, visual and auditory changes, and/or altered states of consciousness.

“Entheogen” is a psychoactive substance that induces alterations in perception, mood, consciousness, cognition, or behavior. The term was coined as a replacement for the terms “hallucinogen” and “psychedelic” but includes other psychoactive substances such as cannabis. All biological entheogens will come with an entourage of co-factors derived from the same source, in the same way as cannabis, cannabinoids, terpenes and flavonoids.

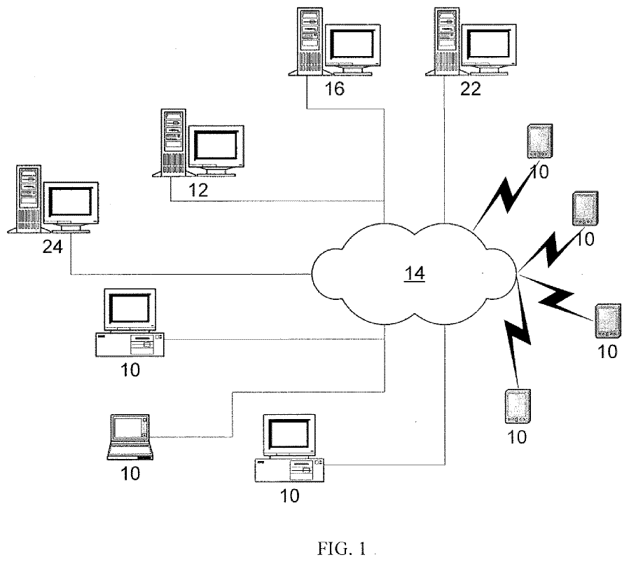

FIG. 1 illustrates a system for digitally standardizing cannabis products. With reference to FIG. 1, the method of the invention is carried out over a computer network such as the Internet 14 by user devices 10, which may be operated by cannabis consumers or producers, comprising a plurality of user computer terminals, whether desktop, tablet, laptop, smart phone, other mobile device or the like. The user devices 10 are provided with application software to access system server 16 via web server 12 and a social network hosting server 24. A machine learning server 22 may also access system server 16 either directly or via the Internet 14.

### A. Internal Graph Curation

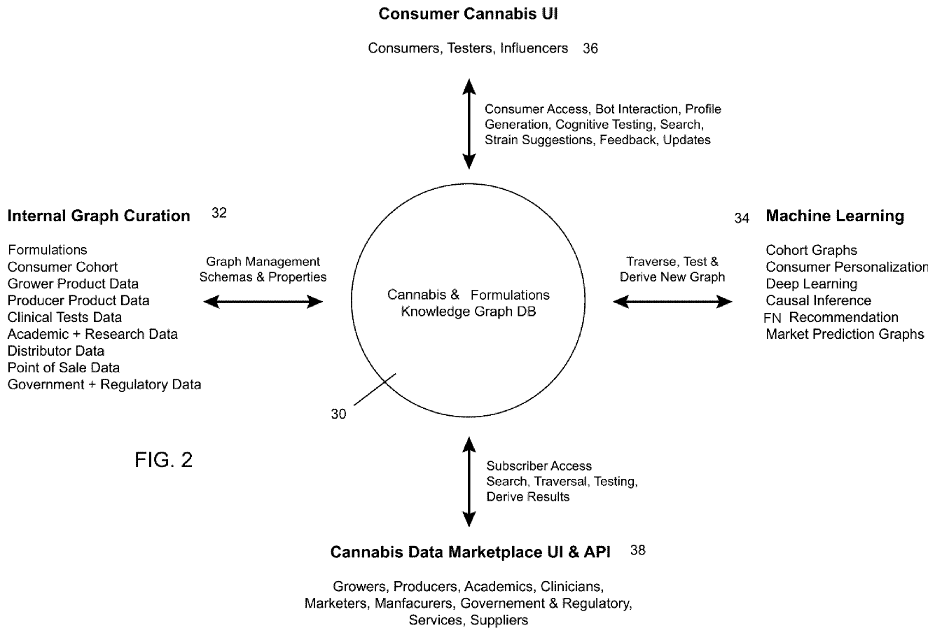

FIG. 2 illustrates the components of the system platform. The Cannabis and Formulations Knowledge Graph database 30, also referred to as the System Platform which may be in the form of a Knowledge Graph Database, may be stored in the system server 16 or as separate databases. The database 30 is curated by the Internal Graph Curation 32, which is responsible for graph management, and creating and defining schemas and properties, which includes storing Formulations, defining Consumer Cohorts, graph management, grower product data, schemas and properties, producer product data, clinical tests data, academic and research data, distributor data, point of sale data and government and regulatory data. Existing Graph DataBase setup and curation may be applied as in the Wikipedia model. There may also be Artificial Intelligence aided ingestion and curation of external cannabis market reports and third party clinical trial data. The Cannabis and Formulations Knowledge Graph DataBase may be built from open source tools like Apache Titan, Cassandra, Kafka and Spark.

The Knowledge Graph is set up first in order for Consumers to use it and interact with the Bot. This entails creating schemas for DataBase objects and their properties. This includes schema for Consumers, Formulations and Cannabis Products.

### 1. Consumer Schema

Looking first at the Consumers, this includes schema for Consumers (users) their details and their private cannabis profiles. For the purposes of the Minimum Viable Product of the platform this is arranged generally as:

Consumer: ([account], [standard], [profile {genetic}, {phenotype}, {health}, {cognitive}, {environment}, {social}])
[Account]: Consumer name, login details, account settings
[Standard]: email, physical address, and the like
[Profile]: The Consumer Profile is everything personal known about the user and may be broken down into six sub-parts:
i) {Genetic}—this may be the results generated by popular ancestry kits such as ‘23 and Me’ kit or Ancestry.com, or other more focused genetic tests for all kinds of DNA queries and more specifically for the pharmacogenetic and cannabis effect.
ii) {Phenotype}—this includes age, sex, height, weight, fitness level, allergies, diseases, eye color, hair color, skin type, many other observable characteristics. This also covers current state as many cannabis users, especially on the medical side, will be looking for specific outcomes to relieve symptoms.
iii) {health}—this incudes the consumer's current health and medical condition, both long-term and short-term (for example, having a cold, arthritis etc.) as might be captured in a consumer's medical/health records.
iv) {Cognitive}—this includes considering pre-existing mental state but also generally the user's behavioral characteristics, mental abilities, IQ, problem solving, physical senses acuity, interests, tastes.
v) {Environment}—details about the user's environment that may affect the cannabis outcome such as altitude, temperature, pressure, pollen count, population density, latitude, longitude, city, travel details, sociological factors and social graph (also described as a separate element below).
vi) {Social} the consumer's social graph, sociological identity, marital status, sexual orientation and other tribal, club, group affiliations and associations.
   The consumer's Profile is structured so that regression tests can be run using Machine Learning algorithms such as ‘K-Nearest Neighbours’ to find statistically relevant correlations to the user's effect and outcome accuracy.

In receiving personal information from users, users will be required to enter an agreement with the System Platform to address privacy and other issues to permit the system to collect such information from the User.

### 2. Formulations (FN) Schema

Each Formulation Profile Schema is made up of four primary parts:
Formulation ({formulation} {delivery} {cohort} {knowledge})
   i) (Formulation): chemical/biological makeup within accurate error margins or as DIGITAL VOLUMES (FNV)—which are defined as multidimensional formulation volumes bounded by accurate FN points.
   ii) (Delivery Vector): Delivery vector would include delivery methods/devices such as smoked, ate, drank, sprayed, vaped and dosage information as accurate as possible.
   iii) (Outcome Cohorts): Outcome Cohorts contain the notions of Effect which is more cognitive in nature and Outcome which is more biological and medical in nature. Outcome Cohorts may be organized by common outcome, genetic similarity, phenotypical similarity (age, sex, fitness and the like), diet, disease, allergies or the like, or all of the above, for a specific experienced effect/outcome. Each Outcome Cohort contains all the users who tried this FN across various different delivery vectors with their reported effects/outcome for each vector.
   iv) (Knowledge & Products): These are links to Certified Products in the Knowledge Graph associated with this FN and other knowledge links for other related data associated with this FN (clinical studies, tests, grower, producer data, chain of authenticity, regulatory and the like).
      By defining Formulations in this way machine learning algorithms can be applied to each FN to look for interesting patterns within a FN itself that might be useful in the Population Cohort studies or elsewhere.

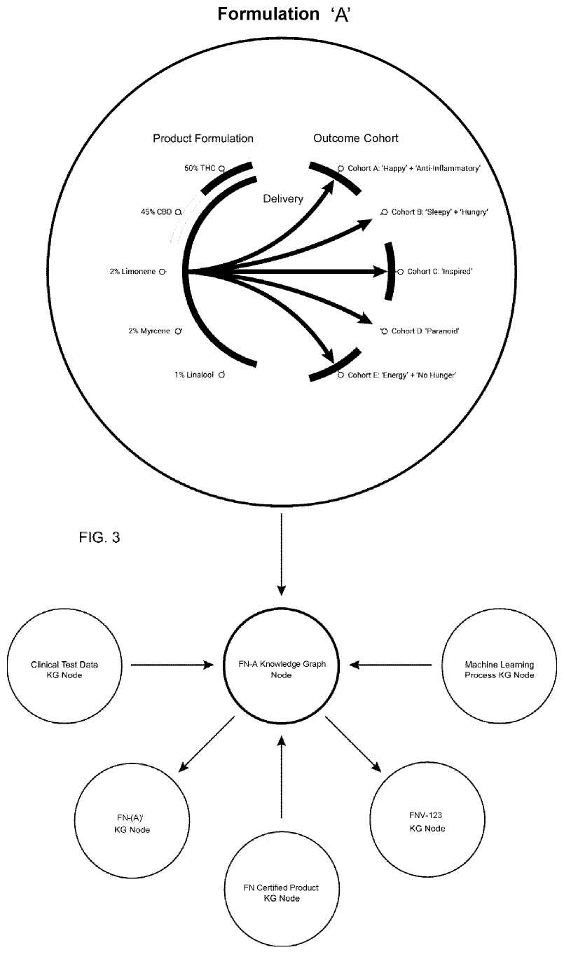

FIG. 3 illustrates an example of a Formulation. As illustrated in FIG. 3, the first stage in standardizing the Formulations is to produce a common set of agreed product formulations, with the goal of producing a universal set of knowns to remove guesswork and provide value to growers, producers, manufacturers, governments and users. The Universal Set of knowns would be created by a mapping of all possible formulations across Cannabinoids, Terpenes and Co-Factors. In the example of Formulation ‘A’ shown in FIG. 3, the formulation is 50% THC, 45% CBD, 2% of the terpene limonene, 2% of the terpene myrcene and 1% of the terpene linalool. Bounded volumes as described under FN Volume below may also create data value in their intersectional overlap with other bounded volumes.

As shown in FIG. 3, for the fixed product formulation in Formulation ‘A’, there are some number of possible delivery methods (smoking, vaping, eating, oils, spray and so on) and for each given method of delivery, data has been generated to identify a large number of cohorts, shown as Cohorts A through E. The subjective outcome of using Formulation ‘A’ on each cohort has been assembled. The effect, for example, of Formulation ‘A’ on Cohort A is happy along with a physical anti-inflammatory effect, on Cohort B is sleepy and hungry, on Cohort C is inspired, on Cohort D is paranoid and Cohort E is energetic and not hungry. Formulation ‘A’ may form a node in the Knowledge Graph with links to the many other nodes in the Knowledge Graph, as illustrated in FIG. 3, including a Clinical Test Data Node, a Machine Learning Node, a node for a related FN Fold Node FN-(A)′ as described below (example, adding alcohol to the formulation components), a FN Certified Product Node representing a producer's specific product, and a FNV-123 Node as described below representing a volume of formulations.

Other Formulation derivatives. Volume (FNV) and Fold (FNF) that take into account the higher dimensionality of the Cannabis Knowledge Graph assist in analyzing and visualizing the space are defined as follows:

FN Volume (FNV)—This is an arrangement of formula discrete FN points that bound a multidimensional volume of formulations. This may allow a Producer to certify a product like Flower or whole plant extract that is complex and difficult to precisely define by exact formulation component ratios. However the Producer may have to choose formulation ranges for components like THC, CBD, Terpenes and other components and thus bound the volume into a discrete boundary solution to then deliver and produce a set of repeatable Outcome Cohorts. However the greater the FN Volume defined, the increasing inaccuracy and decreasing repeatability in Outcome Cohort results. A FNV could also include a FN Fold (FNF) as well.

As an example FN-A above would be grouped with other volume defining FN into a named/labeled ‘FNV-123 (FN-A, FN-B, FN-C, FN-N)’, with its own Delivery Vectors and Outcome Cohorts, that might be designated as a shortened form ‘FNV-123’.
FN Fold (FNF)—In cases where there may be many well-studied FN and FNV sets in the Cannabis Knowledge Graph, additional study may be useful to focus on other specific outcome factors such as levels of caffeine or alcohol to see what happens. While these would be bona fide new Formulations in their own right for notation, testing and visualization, the system can demark these as derived from base FN entities with only one or two new formulation components. This is like a mathematical ‘fold’ of the FN dimensional space to create a visually and computationally simpler FN set. For example FN-A above with caffeine added could be named/labeled as FN-(A)′ with a formulation set of (X % FN-A, Y % Caffeine) with its own Delivery Vectors and Outcome Cohorts. The FNF notation may be used with other various non-Cannabis components like caffeine, nicotine, alcohol and other non-Cannabis derived formulation components. Also an FNV entity could be included in an FN Fold as well. The FNF notation system may be used with any formulation of cannabinoids, terpenes and any other components. It is a method of mapping dimensions down into a lower dimensional ‘fold’ so that humans or Artificial Intelligence can more easily analyze what is going on. Hence it may be used to build optimized Knowledge Graph structure and preserve relationships in Machine Learning and Human facing visualizations, when non typical Cannabis components like caffeine, nicotine, alcohol and other non-Cannabis-derived formulation components are added.

### 3. Cannabis Products Schema

This is used for producers to apply to the platform for certification of a product's Formulation within the System Platform for marketing purposes. A Product Profile is produced by and for the curator as follows, similar to consumer profiles and FN profiles. The Cannabis Product Profile schema defines all of the characteristics for a Cannabis product including Owner Account, Profile formulations, grower location, producer, images, price, and other information that might be included in a marketing brochure.

Product {(account), (profile), (FN certification)}
(Account)—the Producer account to which this Product belongs
(Profile)—All product characteristics including lab tests, Health Canada number and the like
(FN Certification)—a curator assigned FN label/name.

### B. Consumer Cannabis Interface

The Consumer Cannabis User Interface 36 is the primary user interface for consumers (users) to interact with the System Platform 30 and as such, is designed to collect account and profile information from them to help select the desired FN and effect/outcome. Users may consist generally of three types of consumers i) Private. These are Consumers from the general public; these people create accounts to identify suitable cannabis product for their desired outcome. As these consumers provide more Profile information and feedback from the consumption of suggested Formulations, the Platform is able to deliver increasingly accurate product suggestions. ii) Testers. These are people who are paid or otherwise compensated to test different conforming Formulation products and report effects into the system who are tasked to formally fill out the questionnaires with a greater degree of detail, to improve the quality of the data. They may be compensated by loyalty points which can be redeemed for cannabis products. There may be different levels of formalism applied for different testers and trials. Some will be more event driven and colloquial, others very formal and clinical test-like. iii) Influencers. These are people who are paid/compensated to test different conforming FN products and report effects into the system and the social network. The goal is for the consumer in the general public to see the influencers' comments and go to the system platform to sign up and start exploring FN recommendations. Influencers are individuals who influence the general public to follow their lead either due to their status as celebrities or as particularly reliable testers.

Consumer Cannabis User Interface 36 permits Consumer access to the Cannabis and Formulations database 30. Consumers may use the Cannabot Bot User Interface as an automated way of helping users fill out profile data and select Formulation products. Online human agents may also assist where necessary. This may be done in a chat-based User Interface. This may be facilitated by Bot interaction, Search facilitation, Strain Suggestions and Feedback. The Bot may have some artificial intelligence and interactive capability to assist consumers in answering the questions which will establish the consumer's cohort and outcome with particular Formulations. The Consumer's interaction may be through social media applications such as Facebook Messenger, WhatsApp, and the like. The consumer benefits by being provided with accurate prediction of the expected outcome for that individual with particular Formulations and being able to identify suppliers of the desired Formulations.

### C. Machine Learning Interface

The Machine Learning component 34 modifies, tests and updates the Knowledge Graph (“KG”) Database and stores Cohort Graphs, Consumer Personalization Deep Learning, Causal Inference, Formulations Recommendation, and Market Prediction Graphs. Machine learning algorithms traverse the KG and look for patterns. These machine learning algorithms may work directly on individual FN, Population Cohorts, Products or anywhere there could be a correlation of interest to find. There may be a Machine Learning graphs layer with its own schema, objects and properties that has its own edges (links) to FN objects, Consumers, Products or otherwise. Some of the machine learning algorithms which can be used are described in more detail below.

### D. Cannabis Data Marketplace User Interface and API

The fourth component shown in FIG. 2 is the Cannabis Data Marketplace User Interface and API 38. This is where Cannabis businesses would register and curate their Product profiles. It may serve growers, producers, academics, clinicians, marketers, manufacturers, government & regulatory, services, suppliers and the like. The Cannabis Data Marketplace permits subscriber access, search, traversal, testing, and derivation of results. Growers and producers may apply to the system to certify the Formulation for each of their product formulations and delivery methods. This system is of great value to product producers for sales, marketing and prediction and may therefore incent most producers to digitally certify their products. As the platform becomes more formally a data lake (big data), access subscriptions may be offered to industry members to provide/sell their own data, consume the platform's data and run their own Machine Learning tests. The Interface may also be Cannabis business teamware, using a Bot and Human Agent based in a Slack-like UI environment. Application programming interfaces (APIs) may provide the subroutines, communication protocols, and tools for communication between the Cannabis Data Marketplace Users and the FN Knowledge Graph Database. The Cannabis Data Marketplace may provide a marketplace for Industry to buy and sell data and in the future a market for actual FN certified cannabis products using blockchain/Distributed Ledger Technology and asset backed tokens. Since currently biosynthetic cannabinoids like CBD and THC can be created using organic hosts like yeast and sugar and synthetic cannabinoids are made from industrial chemicals, Formulations are a useful tool to standardize the consumer outcome, and may be useful for synthetic and biosynthetic cannabinoids and terpene production, allowing Cannabis producers to recreate a popular FN formulation directly from yeast fermentation methods, as an example.

### Example A: Current Consumer Confusion, Product Selection and Remedy

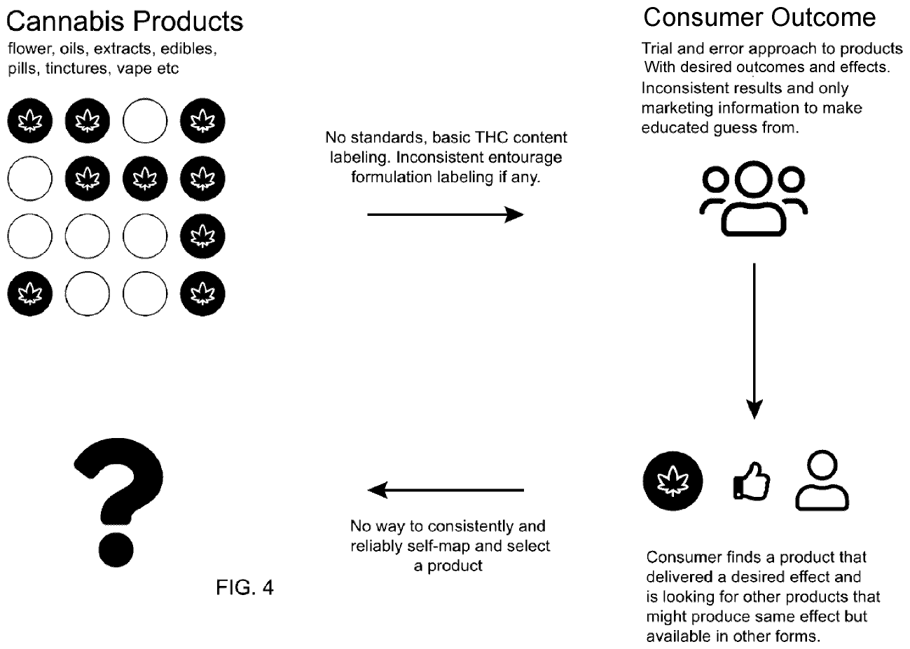

Referring to FIG. 4, the current difficulty in standardizing cannabis products is illustrated. Many different categories of cannabis products are available, such as flowers, oils, extracts, edibles, pills, tinctures, vape and the like, each category having a wide range of formulation accuracy. Currently no standards govern the basic compound formulation labelling on the product. The THC content is inconsistent among sources and has not been properly analyzed, so percentage content is guessed at and is often inaccurate. Similarly the presence of other cannabinoids or terpenes besides THC and CBD has generally not been analyzed or labelled on the product which makes it impossible to assess the likely entourage effect of a given product.

Further, for each of the many different cannabis consumers, whether recreational or medical, each will have very different outcomes with the same product, whether objective or subjective. Consumers have only the suppliers' marketing information to rely on or word-of-mouth. Consequently consumers are forced to make purchasing decisions based on trial and error with inconsistent results. Customers are therefore experiencing difficulty finding certain products that deliver a consistent outcome, are unsure what to purchase and have adopted a very conservative approach to trying new products. If by chance a product is discovered that delivers a desired effect, the consumer will be faced with the same problems if looking to obtain the same desired effect from a different category of cannabis product.

On the part of cannabis producers, in the absence of accurate marketing data they must speculate as to which cannabis products to grow, formulate and sell. Rather they will currently base such decisions on raw sales data and some anecdotal customer feedback. Given the large number of unknowns, the consumer's behavior cannot currently be based on predictable results. The consumer cannot self-map to the appropriate formulation, product category and brand for a desired result. Consequently neither can the consumers' purchasing patterns be predicted by the producers.

### Example B—Application of FN to Solve Current Problem—Consumer Personalization

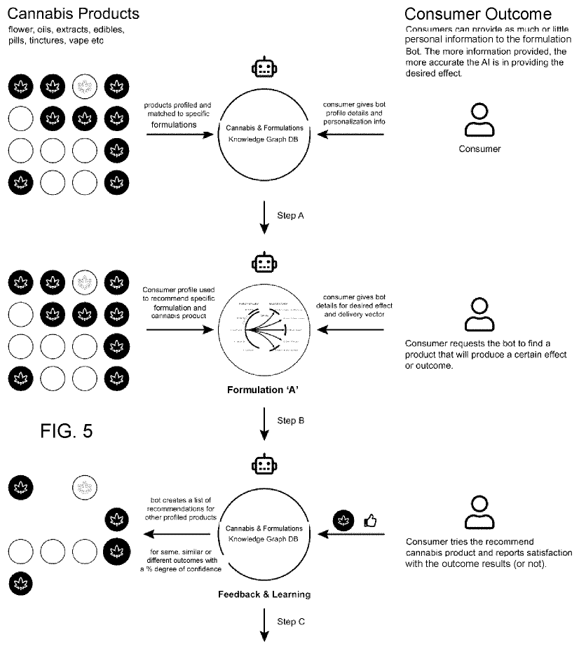

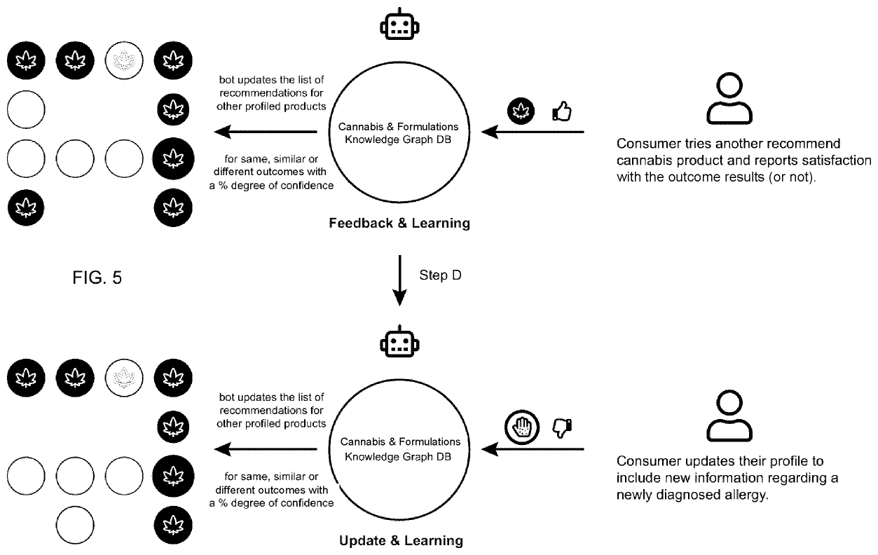

With reference to FIG. 5, there is an illustration of the application of Formulation determination to solve the problem of Consumer personalization.

i) Profile Setup—Once a Consumer or Tester provides at least the most basic profile information, the System Platform is ready to make its first set of predictions. Cannabis Product formulations have been profiled into the FN system by matching to a specific Formulation. This provides a FN certification for the product producer to include in product marketing information. This system is therefore of value to product producers for sales, marketing and prediction which should incentivize most producers to digitally certify their products. A Formulation Bot (“Cannabot”), can then be used by the consumer to identify a desired product by delivery method, effect and outcome, based on the personal information provided by the consumer. Consumers can take a survey test from Cannabot so the consumer can first Cohort self-map and then FN self-map. The consumer may have the option of how much information to provide. Consumers can use less accurate visual cues such as “I resemble person X most from that group”. To obtain more accurate results consumers can provide more information which will allow the Bot/Artificial intelligence to more accurately predict the effect of a given cannabis product on the consumer. A full testing profile may include DNA test results and medical data from health professionals. As Consumers use the system, it becomes more and more accurate. As accuracy increases across the user base, Artificial Intelligence predictive algorithms can provide better data for all users and begin to predict outcomes for cohorts with an acceptable level of probability.
ii) Step A—The Consumer makes a specific request for an effect and possibly a preferred delivery vector. The system searches through the FN set and looks for matching Outcome Cohort information to recommend a FN—in this case “FN-A”.
iii) Step B—The Consumer tries FN-A and reports back to the Bot how it went, if it was as expected or totally different and if so, how. Regardless of feedback, the system can learn from positive or negative results to better tune and personalize the Consumer's profile information for more accurate results.
iv) Step C—The Consumer tries another recommended FN and product. Again the Consumer provides feedback and again the system re-tunes the Consumer profile resulting in a more accurate FN Outcome Set for the Consumer. If a consumer is satisfied with the outcome from a given FN, the consumer can ask the Bot to locate other FN standardized products for the same effect or other different effects. The Bot may then return a list of recommendations for other profiled products for the same, similar or different outcome with a high degree of confidence.
v) Step D—The Consumer updates their profile (phenotype) with some new allergy information and this affects how some terpenes might change the user's outcome, and the system re-adjusts the FN Outcome sets to produce even more accurate recommendations.

### Example C—Using a Precision Medicine Adaptive Study Protocol or Traditional Study Protocol to Set Up and Maintain the User Profile

Traditionally any pharmaceutical drug formulation must go through and pass a statistically significant in vivo testing process to gather results that are then analysed statistically for a pass/fail designation as positive and reliable evidence data for that drugs' outcome efficacy for the target condition of study (e.g. insomnia, pain). These are typically run through a traditional clinical trial process which is built to statistically represent the target population (e.g. all Canadians) by using a carefully selected small subset of testers. The phased results of the trial each have to show statistically significant results including outcome safety and efficacy to pass to the next phase and get approval. Such drug then has become an evidence based formulation.

With the advent of personalized and precision medicine and advances in Artificial Intelligence and computational science, the discovery, analysis and successful testing of evidence based formulations is evolving to treat the individual consumer's genes, body and mind as a knowable single holistic system model that the health system can tailor formulations for the treatment of conditions across medical, wellness and recreational applications. Clinical Trials may now include the creation of an individual's profile for personalized tests of n=1 trials that deliver evidence based formulations for that individuals' needs. Then in aggregate with the personalized results of other individuals, a much more statistically accurate outcome data set of a larger population is created where each new individual can have a profile created and evidence based formulations predicted for the individual's needs by leveraging the aggregate population data set to deliver a greater chance of outcome success.

The following are examples of methods for individuals to contribute experiential data to that User's profile initially in setting up the profile in FIG. 5 and subsequently in steps B and C of FIG. 5. The preferred protocol follows the precision medicine protocol. However the traditional clinical trial protocol may also be useful. The traditional model involves Phase 1-3 studies, with various methodologies comparing one intervention to a group of patients to another. While this helps to establish population data, it is not as effective as precision medicine approaches, i.e. n=1 trials. Traditional research has been designed to be more cost effective to allow for more numbers of users to be enrolled in the trials. However, in today's technological era, there are remote technologies that can allow for n=1 trials, whereby each user will be given personalized attention and the study will be customized and designed for that specific user, with direct expert guidance. The use of AI and machine learning allows for these n=1 trial data to be collated and used to create prediction models based on a given consumers genotypical, phenotypical and cognitive profile.

### I. Precision Medicine Adaptive Study Protocol Overview

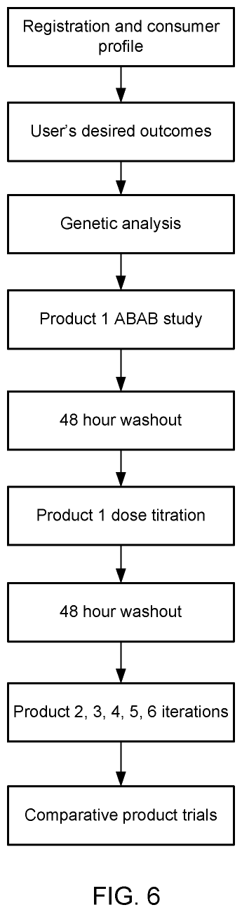

The following is an example of steps undertaken to guide a user through their n=1 adaptive precision research study as shown in FIG. 6.
1. User joins Marijane.ai online platform and is guided towards completing their baseline demographic information through the app; the user completes their consumer profile.
2. The user will indicate the product(s) they currently use and what is their desired outcome, for example THC 5% gel caps for the outcome of sleep.
3. User is invited to complete a genetic analysis through remote throat swab which they will send to the study site.
4. Pre-study Dose Titration: User then participates in a dose escalation/titration study from one of their existing products to determine their minimum effective and maximum tolerated dose, and provides feedback after each dose. They will follow a 4 step dosing protocol, ABCD, where each letter represents an ascending dose of the product. Users will try a product, typically through some sort of inhalational method, and will provide immediate feedback on each dosing level, over a short time frame, such as within 30 minutes. Starting at a low dose, the user will provide a response, and then incrementally increase the dose, and offer feedback on the efficacy as the dose increases. A minimum effective dose, maximum tolerated dose will be determined for each product, dosing method, and connected to this unique consumer profile. For gel caps, the user may for example increase the dose every 24 hours to achieve their desired outcome, such as better quality sleep. Basic reporting data will be collected about whether or not the user achieved the intended outcome and a 1-10 scale of satisfaction with the product.
5. Formal Study: After a 48 hour washout period, the user will then begin a formalized study of a given product (A) at the dose identified in step 4 as producing the minimum effective dose. This product will be utilized on a regular dosing interval for X number of days. After another washout period, the user will then crossover to a placebo/control product. The same data will be collected over X number of days. The cross over again will occur back to product A. Hence the protocol will be ABAB, where A is the active agent and B is the placebo/control substance. Where possible, the patient will be blinded to the product they are using.
5. The same procedure will be completed for iterations of 2^{nd}, 3^{rd}, 4^{th}, 5^{th}, 6^{th} products, etc. Each product will be suggested by the central research site based on expert feedback on chemical compositions, and based on previous data collected from other users with similar profiles.
6. After trialing 6 different products, the user may then participate in comparative studies of 1 product versus another, at the minimum effective dose identified by this user. This will be attempted using blinded methodology to prevent the user having his/her own bias. By comparing and contrasting each product to each other, the user will find the ideal product. The entire time, they will have direct feedback with the host research site. This process will continue for the n=1 trial as long as the user remains engaged and interested in trialing new products. Reporting will be a response on a 1-10 point scale and the user will be prompted by the research site. Again an ABAB cross over design will be used.

### Sample Size:

On a scale of 1-10, it is estimated that an effective cannabis product will produce a mean rating of 6 out of 10. The control group is predicted to produce a rating of 4 out of 10. The standard deviation is predicted to be 3. With 80% power, and 0.05 alpha, this would require a sample size of 36 per group to detect a difference, or a total of 72 subjects. Hence based on the n=1 trials, for a given product, a future Randomized Controlled Trial can use these estimates to calculate the sample size needed to detect a difference. Each product will be tested in an ABAB n=1 trial amongst a minimum of 36 patients prior to making any conclusions about the data. This would help to ensure a reasonable inference of 80% power in this atypical research methodology.

### Statistical Analysis:

Various statistical tests may be utilized corresponding to the study design. If data is collected for 2 or more groups, continuous variable means can be compared using tests such as student's t test or ANOVA. If there are categorical outcomes collected, proportions will be compared using tests such as chi-squared and Fisher's exact test. These statistical tests may be programmed as part of the machine learning and artificial intelligence aspects of the analytical processes.

### Deliverables to the Knowledge Graph:

1. The data from each n=1 study is a useful deliverable which can be input into advanced statistical analysis software and/or Artificial intelligence with machine learning. AI will be able to understand the data and create summative predictive models better than human beings. This data will help the system make consumer specific personalized recommendations. It will also guide the future traditional research studies which might be conducted.
2. The other key deliverable will be information useful for the Cannabis reference guide. The in silico studies will want to be reviewed by health professionals and researchers who will want to know where the data came from and the study design. This will help them understand the quality/rigor of the data collected, i.e. where did the data come from. The adaptive precision n=1 trials will each be summarized and accessible for review.

### Hypothetical Trial Abstract Example:

### Trial ABC123: THC4% for Sleep

Background: A 55 year old Caucasian male was registered and indicated his desired outcome of sleep. He had a prior history of using cannabis and THC gel caps daily for the last 5 years. He lived a balanced lifestyle, working 40 hours a week, working out 3 days a week, and a diverse well balanced diet. His BMI was 28.

Methods: After registration, he also completed a genetic profile. Based on his consumer profile, he was suggested the following 6 products for his initial trials. He began with Product X on a dose titration study. After determining his minimum effective and maximum tolerated dose, he underwent a 48 hour washout phase. He then completed the same process for 5 more products: Products 1 through 5.

Results: For product 1, this minimum effective dose was Xmg, and his maximum tolerated dose was Xmg. He described a satisfaction of 7/10 for this product, with no adverse events. For product 2, this minimum effective dose was Xmg, and his maximum tolerated dose was Xmg. He described a satisfaction of 7/10 for this product, with no adverse events. For product 3, this minimum effective dose was Xmg, and his maximum tolerated dose was Xmg. He described a satisfaction of 7/10 for this product, with no adverse events. For product 4, this minimum effective dose was Xmg, and his maximum tolerated dose was Xmg. He described a satisfaction of 7/10 for this product, with no adverse events. For product 5, this minimum effective dose was Xmg, and his maximum tolerated dose was Xmg. He described a satisfaction of 7/10 for this product, with no adverse events. When conducting comparative trials, he scored highest with product 5.

Conclusion: For this user, the ideal product was 5 with a dosage of Xmg. The second best efficacy was seen with product 2 at a dose of Xmg.

### II. Traditional Study Designs:

The preferred protocol follows the precision medicine protocol as above. However the traditional clinical trial protocol may also be useful for delivering data to the Knowledge Graph. The following trials may be conducted using large groups of users. These methodologies will uncover data and trends in a more traditional methodology.
1. Cohort study—Most studies will follow this format. We will not have a comparative group, but just follow users to collect their data before taking a product, and after taking a product, over a certain time frame. Each cohort study will involve changes to the independent variables.
2. Dose titration study—No comparative group. Users will try a product, typically through some sort of inhalational method, and will provide immediate feedback on each dosing level, over a short time frame, ie within 30 minutes. Starting at a low dose, the user will provide a response, and then incrementally increase the dose, and offer feedback on the efficacy as the dose increases. A minimum effective dose and maximum tolerated dose will be determined for each product, dosing method, and customized to a unique consumer profile.
3. Comparative cohort study—some studies will involve 2 cohorts which should ideally be age and gender matched. One cohort will take 1 product, the other cohort will take a different product. Then we will compare and contrast the results. This could include a comparison of cannabis products to traditional pharmaceutical products.
4. Cross over study—a user will first try product X for a specific time frame. We will collect their data. The user will then try product Y. We will collect the data after using product Y. This will allow each user to compare the response of product X to product Y. One of these products can be a placebo which would be a crossover placebo study. This could include a comparison of cannabis products to traditional pharmaceutical products.
5. Microdose Placebo control—There will be 2 groups of users: 1 group will get the actual effective dose of a product, 1 group will get a microdose product. We will then compare and contrast the results. Ideally the users in the 2 groups are randomized and end up being similar demographics at baseline.
   Blinding: Users do not know which group they are in as the products will look the same. They can purchase 2 different products and then put a sticker on to obscure the label. They can reveal the product after completion of the study. This is possible for only some studies.
   Variables in design: The following independent variables may be changed to conduct different studies: cannabis product, dosage of product, administration method. The key measurables/dependent variables are the outcomes desired, outcomes achieved, negative effects, duration of effect and some of these variables will be measured before usage, within 1 hour of usage, 8-10 hours of usage, and 23 hours after usage.
   Length of studies: There are various potential lengths of each study. For example a study can consist of a single use of a product by X number of users, and hence that would be a 24 hour protocol, with data collected at various time points. Or a study/protocol can last for 7 days, whereby the data is collected over each of those 7 days, and also users can reflect on the experience from day 1 to day 7. Another option of a study length is a 1 month protocol. Ideally studies of various lengths are done to obtain the effect of a product changes with prolonged usage.

### Deliverables to the Knowledge Graph:

1. The data from each study will be a useful deliverable which can be input into advanced statistical analysis software and/or Artificial intelligence with machine learning. AI will be able to understand the data and create summative predictive models better than human beings. However with AI, the important consideration is the quality of the data being inputted. Hence each study should also have a good quality design and raw data records which also demonstrate the trends observed by the AI. AI can guide new protocol designs to fill in data gaps. Nonetheless the majority of the factual understanding of cannabis will come from individual rigorous protocols with raw data which is readily accessible and verifiable.
2. The other key deliverable will be information useful for cannabis formulations reference guide. The in silico studies will need review by health professionals and researchers who will want to know where the data came from and the study design. This will help them understand the quality/rigor of the data collected, i.e. where did the data come from. The following structure may be used for an abstract from one trial with a more detailed report to follow:

### Hypothetical Example Study Abstract

Purpose: To determine the response of users to cannabis product X during 7 days of usage.
Methods: Male and female users from an internal registry were invited to participate in this study. They completed a baseline consumer profile which captured information about their demographics, health, prior cannabis usage/experience, lifestyle, occupation, mood, social situation, environment etc. They were all provided product X which was administered orally at a dosage of Xmg taken once daily at night. The user's provided at baseline their intended outcome from using this product, which was individualized. Self-reported data about the product and its effect was collected at 1 hour after usage, 8 hours after usage, 23 hours after usage. This was repeated each day after each dosage. After 7 days, the users also answered questions about their experience on day 7 as compared to day 1.
Results: There were X males and X females, with a mean age of X. The ancestry distribution was X % white, X % Asian, X % Chinese etc. X % of users had 1-2 years of prior cannabis experience while X % were new users. The most common intended effects described at baseline were: sleep X %, pain relief X %. At 1 hour after usage, X % of users achieved the effect consistently over the 7 days. At 8 hours after usage, X % of users felt comfortable on 80% of days over the 7 days. At 23 hours after usage, X % of users felt comfortable on 80% of days over the 7 days. When comparing the effect between day 7 and day 1, X % of users felt the effect was almost the same and was consistent over time. The percentage of users who achieved the intended effect 80% of the time was X % for pain relief and X % for sleep. The percentage of users satisfied with this product for the 7 day effect of pain relief was X % and for sleep was X %.
Conclusion: Product X used at X dosage over a 7 day period is effective in helping X % users achieve better sleep and X % of users achieve pain relief.

### Example D—Application of the Method as Illustrated by 4 Hypothetical Case Studies Using Cannabis as the Example

As the first step in the process using the System Platform, the set of precise cannabis product formulations is collected and defined. Using modern laboratory extraction, purification and formulation techniques, it is now possible to create discretely and precisely formulated Cannabis products, on the same level of quality as modern synthesized drugs. It is then possible to take a set of precisely formulated Cannabis products, all with the same formulation and map them all into a single designation, for example ‘Formulation X’. This map reduces all products of the same formulation to one known formulation entity and removes the need to try each different commercial product. By applying such a formulation analysis, for example a subset of these commercial products that are truly ‘100% CBD’ may be labelled as ‘Formulation A’.

As the second step in the process an individual's Biological Profile is created, with provision for state updates and curation. A Bot Form interface may be used to begin the individual's Biological Profile. This form obtains answers to questions across genetic, biological and mental factors to complete the profile. Over time this is augmented by notes and checkup updates from a health professional, analytical laboratory test results, wearable health monitoring devices, and an individual's own observations on condition symptom display.

The third step in the process is to create and add knowledge to the Knowledge Graph by using researchers, testers and curators to i) research and create hypothesis (null and alternate) for use in statistical correlation and regression testing; ii) test those hypotheses with real conditions, individuals and cannabis formulations to collect data, statistically analyse for Formulation efficacy and statistically significant biological profile factors that are influential for prediction scoring; iii) build the knowledge graph and annotate it with observations, notes, action items such as suggested tests, actual test results and statistical results; and iv) create a Bot-mediated User Interface to the Knowledge Graph for health professionals and individuals to access the Knowledge Graph for searches and biological profile updates.

The fourth step in the process is to design a drug evaluation condition assay for the individual to ascertain which formulations are the most effective for that individual. A useful Condition Evaluation Assay for Cannabis is composed of 4 Formulations:
Formulation A—100% THC
Formulation B—100% CBD
Formulation C—50% THC | 50% CBD
Formulation D—Placebo

By using each of these in a range of doses with the individual one can determine:
Dosage: microdose, minimum effective dose, maximum tolerated dose
Formulation Variation Efficacy
Basic condition vs formulation efficacy across dosages (regression of efficacy over dosage)
Placebo control.

The fifth step in the process is to facilitate future prediction by creating a scatter plot of the various variables and then statistically analysing them for correlation (r) and significance (p pr p-value). In the following hypothetical case studies a simplified scatterplot of efficacy vs. formulation dosage is considered and then statistically shows that it works for the evaluation assay and determining the influence of biological factors like sex, age and ancestry.

### Use Case 1—Status Quo Selection

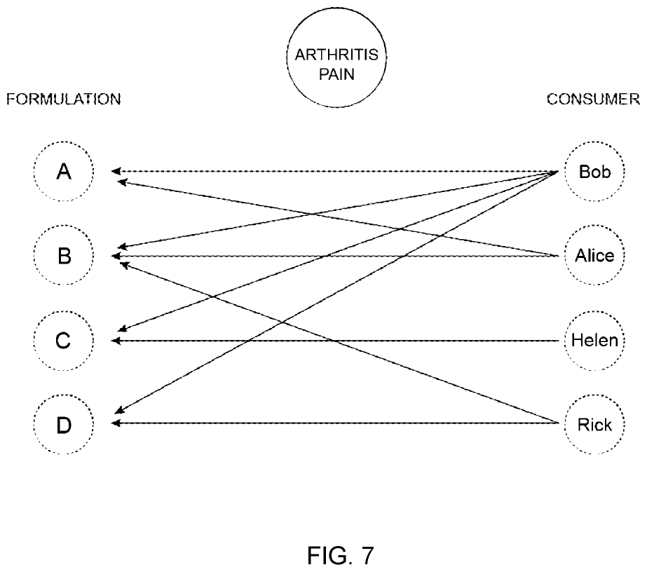

With reference to FIG. 7, four adults, Bob, Alice, Helen and Rick are all looking for relief from their arthritis pain and go to the same online store to purchase a product that might help. Some of them may have done some research online, some may have asked a knowledgeable source, some may simply order something randomly to see what happens and if it works to relieve their pain. None of these people know each other and are all strangers. No personal or health profile information was requested prior to ordering (shown by dashed lines). The online store sells 4 cannabis products, each of which has varying degrees of formulation accuracy on its label (dashed lines) and each is the same delivery vector, an edible.

Bob selects all four products to try and discovers that A works a bit for him but C is much better. B and D didn't do much at all. Alice tries A and B and discover A works. Helen conservatively tries just C but has a negative reaction and does not try any more. Rick tries B and D and neither has any noticeable efficacy for his arthritis, and is discouraged from trying more.

This is a very typical scenario in the real world with people trying to find efficacy by trial and error. Each of these people could write a review of their experience and share on social media but that doesn't guarantee that their outcome will be the same. Helen had an unpleasant reaction to C but it worked for Bob.

### Use Case 2—Map Reduction by Standardization of Formulations

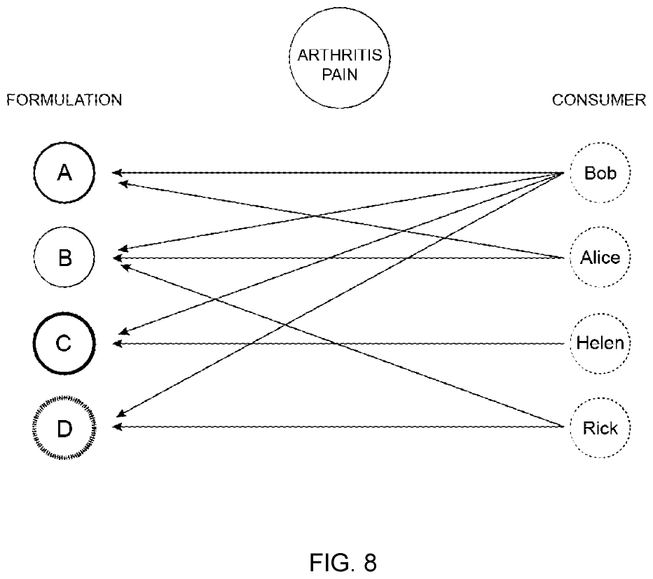

With reference to FIG. 8, in this example, products A, B C and D are required to have exact formulations and that information displayed on their labels and on the online website so all four users can see exactly what is in it. Bob can now see that product A and C have very similar formulations but C is a stronger dose per edible—and that might be the factor in his success. Alice can also see that A and C are similar and may opt to try C now. Helen can see that Formulation C is stronger dosage than all of the others and may opt to try a formulation A with a lesser dose. Rick sees that A and C are a similar formulation than unsuccessful B or D, and may opt to try A or C simply because they are of a different formulation that might be effective.

### Use Case 3—Further Map Reduction by User Profile Standardization

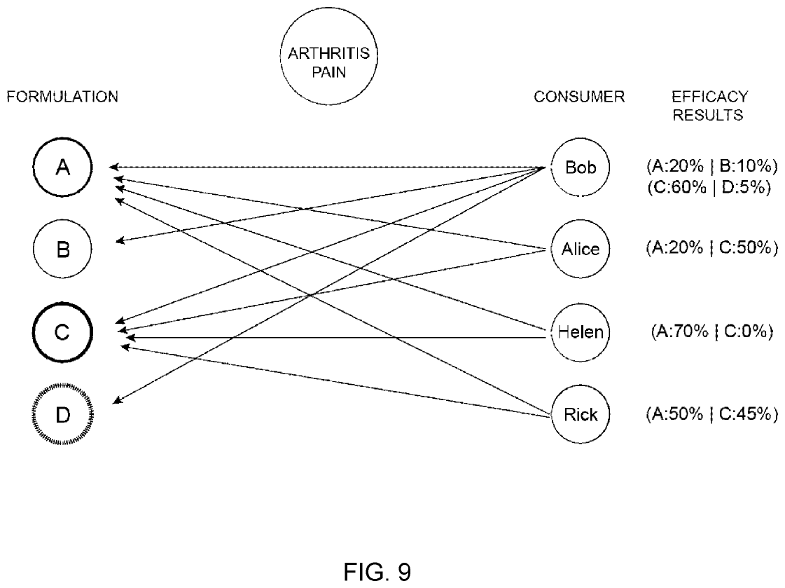

With reference to FIG. 9, in this example there is a set of Testers, each of which came into the system with their own biological profile filled out. There is a statistically significant difference in diversity (for example not all white male millennials) and each Tester has gone through the evaluation assay test and the four online commercial formulations available to Bob, Alice, Helen and Rick. Bob, Alice, Helen and Rick fill out their own Biological Profile form that asks them about their ancestry (approximate genetics), their health details (Age, Weight, Height etc.) and their cognitive states (tired, sleepy, irritable etc.). The anonymized Tester Biological Profiles, Tester Evaluation Assay results and Tester commercial online product results (A,B,C,D) are all made available for Bob, Alice, Helen and Rick to see. As Bob, Alice, Helen and Rick try the various commercial products, their Biological Profile and commercial product efficacy results are anonymized and added to the Tester sets for all to see. So with the right side of the chart now defined as well, they can begin to understand how the product formulations might be affecting different people based on their unique factors. It may also be possible to use statistics to determine if there are any statistically significant profile factor correlations between the Testers and consumers that they share that could be a factor in recommending a formulation to others who share a similar profile feature(s).

So for example Bob (male, 45, 6′, 220 lbs, Caucasian, relaxed) goes first, looks at the Tester commercial product results and tries all 4 products and his efficacy results taken (A:20% | B: 10% | C:60% | D: 5%). Alice (female, 44, 5′5″, 160 lbs, Caucasian, tired) investigates the Tester product results and chooses a few of the female Testers that seem to have some profile similarities to herself and determines that formulation A or C might be a better bet to try. Alice tries A and C and has significantly better efficacy results than “Buy and Try” random selection—an improvement. Helen (female, 28, 120 lbs, Asian, energetic) investigates the Tester product results and chooses a few of the female Testers that seem to have some profile similarities to herself. She has a good efficacy result from formulation A but formulation C is simply too strong a dosage. Rick (male, 33, 180 lbs, Asian, calm) sees the Testers results and chooses to try higher male efficacy results. He decides that sex and race may be key factors in efficacy. He gets good results from both and is benefitting greatly by the use of standardized formulation and consumer profiles. Statistically, this creates a better method to help people choose, as more people use the system, the easier it will be to get the benefit of a shared efficacy determining factor in product selection.

### Use Case 4—Use of Statistics to Define Profile Feature Cohorts and Recommendation Scores

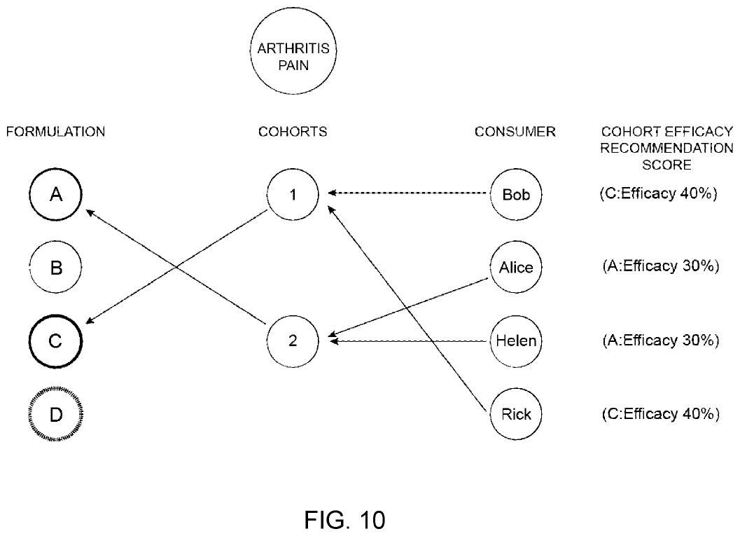

Finally with reference to FIG. 10, there is a set of Testers, each of which came into the system with their own biological profile filled out. There is a statistically significant difference in diversity (for example not all white male millennials) and each Tester has gone through the evaluation assay test and the four online commercial formulations available to Bob, Alice, Helen and Rick. Bob, Alice, Helen and Rick fill out their own Biological Profile form that asks them about their ancestry (approx. genetics), their health details (Age, Weight, Height etc) and their cognitive states (tired, sleepy, irritable etc.). Again the anonymized Tester Biological Profiles, Tester Evaluation Assay results and Tester commercial online product results (A,B,C,D) are all made available for Bob, Alice, Helen and Rick to look at.

As Bob, Alice, Helen and Rick try the various commercial products, their Biological Profile and commercial product efficacy results are anonymized and added to the Tester sets for the others to see. Knowledge aggregates and accumulates. But instead of giving them other consumer's profile to sift through to find their own matches—the system is graphing (product vs. efficacy) vs (sex, age, race, mood) as it goes along, deriving statistical correlations and regression. The statistics shows that across all Testers, for treating arthritis pain with these four products, statistically there are two distinct Cohorts based on the profile attributes (sex | weight). The system sets a Cohort Recommendation score that is the mean value of the results from the user's (or other's) previous tests and gives each user its formulation cohort scores. It is likely that different Conditions will create different Profile Factor Cohorts and will change membership as each condition will be significantly affected by different biological profile factors.

As Bob and Rick are both the same sex and similar in body weight, they belong to Arthritis Pain Profile Factor Cohort 1, which for these set of products, statistically formulation C has a mean recommendation efficacy score of 40%. As Alice and Helen are both female and similar in body weight, they belong to Arthritis Pain Profile Factor Cohort 2, which for these set of products, statistically formulation A has a mean recommendation efficacy score of 30%.

In research to date the applicant has discovered that the major cohort correlations with r>0.5 and p value<0.05 are for (sex, age and DNA). With more testers and products, It is possible for a consumer to belong to more than one Cohort at a time. With more testers and products, It is possible for a Cohort to indicate (point to) more than one formulation at a time. Both of the above could indicate that Cohorts may have sub cohorts or there are other factors that could be broken out into related but separate Cohorts with non-overlapping members
In sample tests ABCD were:
5 mg THC
5 mg THC | 5 mg CBD
5 mg CBD
Placebo

The foregoing cases illustrate how the application of the disclosed method to cannabis can provide Evidence-based Formulations with a statistical outcome efficacy proof of p<0.05 statistical significance from Tester data (N=10, N=100) that proves statistical significance vs, placebos and other formulation efficacy. This can be the basis for standardized Evidence-based Formulations.

### Machine Learning

As the Customer, FN and Product profiles begin to fill up the KG DB, a plethora of different Machine Learning algorithms (ML) may operate to conduct data mining, analysis and prediction. This may include methods like the following:
K-Means Clustering, which looks to find group partitions in the FN Outcome Cohorts that would indicate that certain Consumer Profiles belong in certain partitioned groups with a common response to the FN formulations.
Deep Learning through Linear Regression, which looks to model Consumer and Population level Outcome Cohorts to understand the statistical likelihood of a Consumer Profile mapping to Outcome Cohorts across one or many related FN formulations.
Deep Learning through Neural Networks, Recurrent Neural Networks (RNN), Convolutional Neural Networks (CNN) and Deep Neural Networks (DNN), which look at data pattern recognition spread more broadly across Product, FN and Consumer profiles, to predict what Consumer choice preferences might gravitate to under different sets of market conditions and FN formulations.
Multi-Grained Cascade Forest (gcForest), which looks at live video of Consumers for baseline cognitive tests to pick out speech, movement and personality features unique to that individual to see how they change with FN product consumption and/or ethnic, sex and phenotypic face and body profiling to better map the Consumer's genotype and phenotype properties.
Causal Inference, which looks to predict what causes Consumers to choose certain Outcome Cohort effects and outcomes to better match their personalization profiles to factors that can be inferred like environment change (allergy seasons), Social planning (event type driven consumption) and others.

One of the above methods may traverse the Knowledge Graph FN, through Product, Consumer and FN profiles, using custom schemas to create new graphs of statistically probable relationships. Hence new knowledge may be formed by automated ML graph traversal for human evaluation and consideration. Each new graph ML traversal can create Graph Derivatives data products like graphs (such as line graph), charts (such as chord charts) and multi-dimensional volume visualizations on which human researchers can collaborate. In silico testing may be used to create a synthetic consumer profile simulation and model that entity's Outcome Cohort results, to use the simulation to replace the need for constant human FN testing, similar to Protein folding prediction algorithms where the computer can ascertain a protein's properties simply from its amino acid sequence. Thus with constant user feedback and updates (as described above) previous ML methods can be re-run to increase accuracy and fine tune results. The Machine Learning part of the platform thereby begins to suggest and predict when certain FN's are going to be needed in the market place and by whom, to predict when there needs to be more research regarding suspected new biological processes in humans (such as the potentially new CB receptor) and what new FN formulations should be made and tested that would confer highly desired Effects/Outcomes across a few or many different Consumer Profiles, such as where ML may predict a new popular FN formulation that does not yet exist.

### Cannabis Data, Service & Token Marketplace

From the opposite perspective from Consumer UI and profiles, the Industry concern is to demonstrate how they benefit from Formulations as compared to what they have now and ways they can use it to get back critical data for product configuration/formulation and marketing data. The Cannabis Data Marketplace represents how business teams across Growers, Producers, Academics, Clinicians, Marketers, Government, Services, Suppliers, and Cosmetics and Health Professionals can collaborate within their own organizations and across other organizations to use the Knowledge Graph and its data to gain a better understanding of their goals in the Cannabis industry. Growers and Producers may benefit from FN certifying their products. This primarily centers around the fact that standardization will inevitably be required in some form and consumers will only be able to trust cannabis products that are known formulations and delivery dosages. Doctors and health practitioners need this standardization to prescribe cannabis products with the knowledge that the products will work to achieve the exact effects and outcomes needed taking into account the difficulties of the personalized cannabis consumer outcome. Value is provided right away to consumers by the disclosed method increasing the likelihood of the consumer getting desired effects and outcomes. This has value to all sectors of the Cannabis market.

Using the disclosed method Growers and Producers can leverage FN Formulation/Delivery/Effect Cohort data to tune, configure or create new products, even if producers don't certify their products. Growers and Producers may sponsor/advertise specific Formulations and their linked certified products and provide a way to guide the consumer to order those via a link out to an online store. Anyone can buy, sell or advertise in the Cannabis Marketplace for data products and/or services. The Cannabis Marketplace provides a medium for cannabis product exchange where Cannabis products are traded in real time. The FN Certification system enables this as it specifies exactly what is being sold/bought, in real time. Currently there is no valid cannabis exchange marketplace that can guarantee exactly what is being traded. Once commodities are FN certified, like a stock, one can invest in a cannabis commodity through the commodity exchanges. One can also do commodity trading using futures contracts or derivatives. The FN certified cannabis commodities market as described above may operate just like any other market. It is a physical or a virtual space, where one can buy, sell or trade various commodities at current or future date. Marketplace users may also use the Bot to help them search, traverse and run ML tests on the KG.

There may also be provided a product certification service that includes the ability to add specific FN Iconography for consumers to visually identify how that FN affects them. This may be achieved for example by using emoji or other graphics to augment or replace the exact FN labels. For example ‘FN-A’ could be represented by a winking emoji. There may be a product certification service that includes the ability to add a specific QR code or matrix barcode or link to direct the user to the correct product FN information for example ‘mjane.im/dsa123’. The user may be able to pull up FN certified product information from the package in the store by using augmented reality on their smartphone, thus displaying information hovering above or on top of the cannabis product package.

Blockchain or digital ledger technology may be added to any FN certified cannabis commodity in the marketplace. This enables the ‘crypto tokenization’ of FN certified products which is immutable and can provide proof of origin, proof of chain-of-custody and futures smart contracts on Ethereum. Also ‘crypto asset backed cannabis tokens’ may be traded openly much like the gold-backed crypto currency stablecoins or US dollar backed stablecoins, for example a CBD backed Cannabis stablecoin that would be fixed to the value of one litre of pure CBD (‘FN-CBD’ certified). As described above, Formulations are not constrained to Cannabinoids and Terpenes. FN formulations may include other non-Cannabis products namely psychedelics, entheogens and other bioactive molecular formulations, including caffeine, alcohol, nicotine, flavonoids or more traditional pharmaceuticals such as ibuprofen, aspirin and the like, which expands the current market possibility for product variety and transactional growth. By subscribing to various configurations and packages of data, services, tokens and ML results in the Marketplace, every sector of the Cannabis market benefits.

### Internal Graph Curation

### Structured and Unstructured Data Curation

The following are examples of how the FN Knowledge Graph may be curated. Schema will be developed or used from open source to fill the KG with more information from 3rd party Clinical tests, grower data, producer, distributor or any other kind of relevant data that might be useful and/or referred to by FN Product or KG link properties.

### Formulation Results Curation

For FN Formulation, the pre-curated search set is all FN profiles across all Formulations, Folds and volumes. Focused and specific machine learning algorithms are run on this as a whole to identify interesting patterns that may help users self-identify and select products. This is a separate graph of ‘Interesting FN's’, where the multidimensional points are all known formulation components. For example a FN graph might be all FN, FNF, FNVs that contain limonene and turmeric.

### Population Cohorts Results Curation

For Effects and Outcomes, the schema for the hashed private set of all user profiles across all tracked effects are arranged into Cohort-per-effect, ethnography, sex, age or any other major factor. Focused and specific machine learning algorithms may be run on this as a whole to identify interesting patterns that may help users self-identify and select products. This is a separate graph of Cohorts, similar to the FN, where the multidimensional points are effects and outcomes. For example the pattern detected may be that everyone who has smoked these Formulations was asleep in 15 minutes or less.

### Delivery Vector Results Curation

For Delivery Vector curation, the schema for the set of all Delivery Vectors may be mapped to Population Cohort Effects and Outcomes. Focused and specific machine learning algorithms may be run on this set as a whole to identify interesting patterns that may help users self-identify and select products. This is a separate graph of Cohorts, similar to the FN, where the multidimensional points are Delivery Vectors mapped to effects and outcomes. For example the pattern detected may be the set of all Delivery Vectors who had a positive arthritis pain reduction outcome.

### Synthetic and User Avatar Curation

A Consumer Profile may be made up, much as is currently done on Instagram, of a non-existent ‘synthetic’ user for people to self-identify with for “I'm like that guy” Consumer Profile data collection. While not precise, more information may be collected more quickly from the user by making it easy for them just to look at a number of images of synthetic users and pick a few that they are like in terms of ancestry, sex, body type, age etc. The user may also be presented with an easy-to-navigate avatar UI where they can choose their avatar's ethnicity, sex, age, body type.

### Consumer Cannabis UI

While a large part of the current problem is the extreme variability in Cannabis product formulations, people's profiles themselves are also a moving target with many unknowns. The Consumer Cannabis UI helps people profile themselves so the platform can help them find what they need from the Formulation system. However people change over time with age-related processes, diet, new environments, allergies and many other things. The Consumer Cannabis UI may update information from the user without expecting them to constantly fill out forms, which is unrealistic. This requires constantly learning new things about the consumer and also using feedback from product recommendations to learn, adapt and fine tune the recommendation system for more accurate results. This may be assisted by the following:
A friendly ‘Cannabot’ interface that runs in different messaging platforms like Facebook Messenger, Snapchat, Instagram, Whatsapp, SMS and other social networking messaging platforms.
The Bot helps the user to build the profile in as few steps as possible, using forms, images and media to help them provide information
The Bot may use, with the user's permission, facial recognition to determine genetic, phenotypic, location and other variables.
The Bot may guide the user through the ‘I'm like that guy’ system of visual self-profiling by displaying arrays of real or synthetic user for the consumer to scroll through to find one that they feel is close to their own ethnicity, age, sex etc.
Once the Bot has enough initial profile data, it will begin asking the user what their preferred Cannabis delivery vectors are and the desired effects and/or outcomes.
The Bot may also administer cognitive tests in the form of questions or games to get a baseline cognitive profile of the user prior to product consumption. The Bot may do further tests post-consumption to gather more cognitive effect information for the consumer's profile.

The more that is known about the consumer, the closer the AI can pattern match to a cohort and estimate likelihood the user will achieve a desired effect. Matching the consumer to the right FN is difficult, but delivering close to the desired effect as possible, even as a statistical percentage of accuracy, is an improvement on the current unpredictably in cannabis product selection. The more known about the particular consumer, the higher confidence value that can be delivered. An Outcome Cohort can be a cohort of one, as many will indeed start that way. Using the Cannabot to administer cognitive test and games to set a ‘baseline’ behavior profile and then test the user after Delivery Vector to measure not only the Effect/Outcome but other things like memory, reaction time and other factors will help achieve that. The Consumer may be sent a test sample kit (as would any tester or influencer) with FN certified products in it to consume, report and create an initial product profile map. The Consumer may be sent these profiling kits either stock or personalized to further hone their profile and Outcome Cohort data. Facial Recognition for Consumer profile can help other Users with profile Effect Cohort overlap and regression whereby the Bot may use facial recognition technology to match the user to a cohort. The Consumer profile Bot/UI may thereby allow someone to self-identify with someone (real or synthetic) to help determine profile {genetics} {phenotype} {cognitive} {environment} (I'm like that guy). Consumers can provide full testing profile by including DNA test results and medical data from health professionals. As a Consumer uses the system, the system becomes more and more accurate. As accuracy increases across the user base, AI predictive algorithms can provide better data for all users and begin to predict outcomes for cohorts with an expected level of probability. The end goal is to provide Consumer Personalization with Cannabis Product selection by recommendation, constant feedback and profile updates.

People's cognitive subjective usage of source changes throughout their lives and across many different co-factors. While generally there is a basic knowledge of ‘this source is likely to give me some kind of THC buzz’ or ‘this is CBD so it may relax me or make me drowzy’, there is currently only anecdotal, poorly-studied evidence for dosage, ‘The Entourage Effect’, alcohol, caffeine and genetics. People may directly consume Formulation dosages to record subject cognitive outcome along with knowledge about co-factor influences. In this way, AI may comb this data to look for patterns and suggest new Formulations to consume to identify the Formulations to deliver the exact desired outcome for that individual. This facilitates the cannabis subjective outcome by personalizing the outcome around the individual while also providing anonymized, aggregated data value for other users who share co-factor traits.

Source producers may create ISO bounded volume formulations indicated on packaging at point-of-sale for users. These formulations may be tracked over the life of the plant to products for verification. FN ISO formulations may be traded on blockchain like future or commodities. People's data may be anonymized and shared as data on blockchain. From this the individuals may be compensated directly and anonymously for their data contributions using personalized loyalty points, thus incentivizing the contributors to continue to contribute data capturing deeper and richer co-factor and genetic knowledge.

Outcome Cohorts may be organized by common outcome, genetic similarity, phenotypical similarity (age, sex, fitness and the like), diet, disease, allergies and the like, or all of the above. Each Formulation carries with it at least product chemical formulation and delivery methods and dosage, joined by the effect delivery details. For a known formulation, known delivery and known (to the best possible level) consumer cohort, prediction of the consumer outcome may reach suitable accuracy. The FN system may issue each consumer a code that tells the producer store what the consumer's cohort is to map from a given cannabis product with a given FN. If brands do not want to display FN codes on products they can use other graphical codes to display the FN. The more that is known about the specific consumer, the closer the Artificial Intelligence can pattern match to a cohort and estimate likelihood the consumer will achieve desired effect for a given FN. As other cohort members fill in unknowns, this delivers better cohort or herd outcome to all consumers by providing reproducible results. The more that is then known about the particular consumer, the more accurate the determination of that consumer's cohort will be and the higher the accuracy of the predicted effect by a given FN that can be delivered. A Bot can be used to collect this information for an informal cohort match and the more the consumers give the Bot, the higher the likelihood of success. A Cohort can be a cohort of one, or a user can be in more than one cohort.

Formulations (“FN”) may be used as a testing reference point to map not only exactly to an existing FN formulation but also into known, bounded volumes (ie, [FN-A, FN-B, FN-C]. Used as exacting reference points, it will be easier and cheaper and far more accurate and reproducible outcomes for source producers to use the FN system to test against, rather than getting people to simply report their outcome. The foregoing system platform therefore provides i) Formulations as Certifications for cannabis products and cohort effect guarantee; ii) tokenization of FN certified products; iii) certification for asset backed cannabis products which need certification; and iv) Iconography or QR code so a user can be quickly directed to FN information.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

1. A computer-implemented method for generating a bioactive product recommendation for a consumer, the method comprising:
receiving a consumer profile and a desired outcome from a consumer, wherein the consumer profile comprises one or more genetic factors, one or more phenotype factors, and one or more cognitive factors;
receiving a set of tester profiles, a set of bioactive formulations, and a set of tester outcomes, wherein:
each of the tester profiles comprises one or more genetic factors, one or more phenotype factors, and one or more cognitive factors;
each of the bioactive formulations comprises a delivery vector and a set of bioactive molecules and corresponding dosages;
each of the tester outcomes is associated with one of the tester profiles and one of the bioactive formulations;
determining one or more profile similarities between one or more genetic factors, phenotype factors and cognitive factors of one or more tester profiles and one or more genetic factors, phenotype factors and cognitive factors of the consumer profile;
generating a bio-similar tester cohort based on the profile similarities;
determining a recommended bioactive formulation based at least in part on the bioactive formulations and the tester outcomes associated with the bio-similar tester cohort;
receiving a set of bioactive products, wherein each of the bioactive products comprises a delivery vector and a set of bioactive molecules and corresponding dosages;
identifying a closest bioactive product to the recommended bioactive formulation based on the delivery vector and set of bioactive molecules and corresponding dosages of the recommended bioactive formulation and the delivery vector and set of bioactive molecules and corresponding dosages of the bioactive products;
generating a confidence interval based in part on the recommended bioactive formulation, the closest bioactive product and the profile similarities; and
providing the confidence interval and a bioactive product recommendation comprising the closest bioactive product to the consumer.

2. The method according to claim 1, wherein each of the tester outcomes comprises an efficacy, and generating the confidence interval comprises generating the confidence interval based in part on the efficacy of the tester outcomes.

3. The method according to claim 1, wherein the set of tester profiles includes a tester profiles with a statistically significant range of genetic factors, phenotype factors and cognitive factors.

4. The method according to claim 1, wherein:
the consumer profile comprises one or more health factors, one or more environmental factors, and one or more social factors;
each of the tester profiles comprises one or more health factors, one or more environmental factors, and one or more social factors; and
determining the one or more profile similarities comprises determining one or more similarities between one or more health factors, environmental factors and social factors of one or more tester profiles and one or more health factors, environmental factors and social factors of the consumer profile.

5. The method according to claim 4, wherein:
the desired outcome corresponds to one of the health factors or cognitive factors of the consumer profile;
each of the tester outcomes corresponds to one of the health factors or cognitive factors of one of the tester profiles;
determining the one or more profile similarities comprises determining at least one similarity between the health factor or cognitive factor associated with the desired outcome and the health factor or cognitive factor associated with each of the tester outcomes; and
determining the recommended bioactive formulation comprises determining the recommended bioactive formulation based at least in part on the at least one similarity between the health factor or cognitive factor associated with the desired outcome and the health factor or cognitive factor associated with each of the tester outcomes.

6. The method according to claim 1, wherein determining the recommended bioactive formulation comprises determining the recommended bioactive formulation with a machine learning algorithm trained on the tester profiles, bioactive formulations, and tester outcomes.

7. The method according to claim 1, wherein receiving the consumer profile comprises generating the consumer profile.

8. The method according to claim 7, wherein generating the consumer profile comprises receiving one or more answers to one or more consumer survey questions.

9. The method according to claim 8, wherein generating the consumer profile comprises receiving one or more outcomes and associated test product formulations from the consumer.

10. The method according to claim 9, wherein generating the consumer profile further comprises providing the test product formulations to the consumer.

11. The method according to claim 10, wherein providing the test product formulations to the consumer comprises generating the test product formulations at least in part based on the one or more answers to the one or more consumer survey questions.

12. The method according to claim 1, further comprising providing one or more bioactive product reviews to the consumer, wherein providing the one or more bioactive product reviews to the consumer comprises:
receiving a set of bioactive product reviews, wherein each of the bioactive product reviews is associated with one of the tester profiles;
selecting one or more of the bioactive product reviews associated with a tester profile in the bio-similar tester cohort; and
providing the one or more selected bioactive product reviews to the consumer.

13. The method according to claim 1, wherein the delivery vector includes one of: smoking, eating, spraying, and vaping.

14. The method according to claim 1, wherein the bioactive product is selected from the group comprising cannabis products, psychedelics products and entheogen products.
